(19) United States
(12) Patent Application Publication
(10) Pub. No.: US 2007/0032404 A1
(43) Pub. Date: Feb. 8, 2007

(54) Methods for treating diabetes and related disorders using pde10a inhibitors

(75) Inventor: Laurel Sweet, Branford, CT (US)

Correspondence Address: JEFFREY M. GREENMAN, BAYER PHARMACEUTICALS CORPORATION, 400 MORGAN LANE, WEST HAVEN, CT, 06516, US

(73) Assignee: Bayer Pharmaceuticals Corporation, West Haven, CT (US)

(21) Appl. No.: 10/564,680

(22) PCT Filed: Jul. 27, 2004
(86) PCT No.: PCT/US04/24073

§ 371 Date: Jan. 13, 2006

Related U.S. Application Data
(60) Provisional application No. 60/491,730, filed on Jul. 31, 2003.

(51) Int. Cl. A61K 38/28; A61K 31/7034; A61K 31/519; A61K 31/155; A61K 31/426; A61K 31/175

(52) U.S. Cl. 514/3; 514/25; 514/252.16; 514/262.1; 514/369; 514/592; 514/635

(57) ABSTRACT

The methods of the invention relate to the treatment of diabetes, including type 2 diabetes, and related disorders by administration of a PDE10A inhibitor. Such PDE10A inhibitors may be administered in conjunction with alpha-glucosidase inhibitors, insulin sensitizers, insulin secretagogues, hepatic glucose output lowering compounds, B-3 agonist, or insulin. Such PDE10A inhibitors may also be administered in conjunction with body weight reducing agents. Further methods of the invention relate to stimulating insulin release from pancreatic cells, for example, in response to an elevation in blood glucose concentration, by administration of a PDE10A inhibitor.

This application claims benefit of U.S. Provisional Application Ser. No. 60/491,730, filed on Jul. 31, 2003, the contents of which are incorporated herein by reference in their entirety.

### FIELD OF THE INVENTION

The invention relates to methods of treating diabetes and related disorders by administering a compound that inhibits PDE10A. Such PDE10A inhibitors may be administered in combination with other pharmaceutical agents, for example, anti-diabetic agents or body weight reducing agents. Further methods of the invention relate to stimulating insulin release from pancreatic cells, for example, in response to an elevation in blood glucose concentration, by administration of a PDE10A inhibitor.

### BACKGROUND

Diabetes is characterized by impaired glucose metabolism manifesting itself among other things by an elevated blood glucose level in the diabetic patient. Underlying defects lead to a classification of diabetes into two major groups: type 1 and type 2. Type 1 diabetes, or insulin dependent diabetes mellitus (IDDM), arises when patients lack insulin-producing β-cells in their pancreatic glands. Type 2 diabetes, or non-insulin dependent diabetes mellitus (NIDDM), occurs in patients with impaired β-cell function and alterations in insulin action.

The current treatment for type 1 diabetic patients is the injection of insulin, while the majority of type 2 diabetic patients are treated with agents that stimulate β-cell function or with agents that enhance the tissue sensitivity of the patients towards insulin. The drugs presently used to treat type 2 diabetes include alpha-glucosidase inhibitors, insulin sensitizers, insulin secretagogues, metformin, and insulin.

Over time, more than one-third of all type 2 diabetic subjects lose their response to oral agents. Insulin treatment is instituted after diet, exercise, and oral medications have failed to adequately control blood glucose. The drawbacks of insulin treatment include, for example, the need for drug injection, the potential for hypoglycemia, and weight gain.

Another strategy for diabetes therapy is based on the cyclic adenosine monophosphate (cAMP) signaling mechanism and its effects on insulin secretion. Metabolism of glucose promotes the closure of ATP-dependent K⁺ channels, which leads to cell depolarization and subsequent opening of Ca⁺⁺ channels. This in turn results in the exocytosis of insulin granules. cAMP is a major regulator of glucose-stimulated insulin secretion. The effect of cAMP is, however, glucose-dependent, that is, cAMP has little if any effects on insulin secretion at low glucose concentrations (Weinhaus, et al., Diabetes 47:1426-1435, 1998). The effects of cAMP on insulin secretion are thought to be mediated by a protein kinase A pathway.

Endogenous secretagogues utilize the cAMP system to regulate insulin secretion in a glucose-dependent fashion (Komatsu, et al., Diabetes 46:1928-1938, 1997). Examples of such endogenous secretagogues include pituitary adenylate cyclase activating peptide (PACAP), vasoactive intestinal polypeptide (VIP), and glucagon-like peptide-1 (GLP-1).

PACAP is a potent stimulator of glucose-dependent insulin secretion from pancreatic β-cells. Three different PACAP receptor types (R1, R2, and R3) have been described (Harmar, et al., Pharmacol. Rev. 50:265-270, 1998). The insulinotropic action of PACAP is mediated by the GTP binding protein Gs. Accumulation of intracellular cAMP in turn activates nonselective cation channels in β-cells increasing [Ca⁺], and promoting the exocytosis of insulin-containing secretory granules.

Vasoactive intestinal peptide (VIP) is a 28 amino acid peptide that was first isolated from hog upper small intestine (Said and Mutt, Science 169:1217-1218, 1970; U.S. Pat. No. 3,879,371). The biological effects of VIP are mediated by the activation of membrane-bound receptor proteins that are coupled to the intracellular cAMP signaling system.

GLP-1 is released from the intestinal L-cell after a meal and functions as an incretin hormone (i.e., it potentiates glucose-induced insulin release from the pancreatic β-cell). It is a 37-amino acid peptide that is differentially expressed by the glucagon gene, depending upon tissue type. The clinical data that support the beneficial effect of raising cAMP levels in β-cells have been demonstrated with GLP-1. Infusions of GLP-1 in poorly controlled type 2 diabetics normalized their fasting blood glucose levels (Gutniak, et al., New Eng. J. Med. 326:1316-1322, 1992) and with longer infusions improved the β-cell function as compared to normal subjects (Rachman, et al., Diabetes 45:1524-1530, 1996). A recent report has shown that GLP-1 improves the ability of β-cells to respond to glucose in subjects with impaired glucose tolerance (Byrne, et al., Diabetes 47:1259-1265, 1998).

The use of such endogenous secretagogues to treat type 2 diabetes also has some drawbacks. For instance, the peptidyl nature of these compounds requires that they be administered by injection. Additionally, the effects of the endogenous secretagogues are short-lived because of the short half-life of the peptides.

Because of the problems with current treatments, new therapies to treat type 2 diabetes are needed. In particular, new treatments to maintain normal (glucose-dependent) insulin secretion are needed. Such new drugs should have the following characteristics: 1) dependency on glucose for promoting insulin secretion, that is, compounds that stimulate insulin secretion only in the presence of elevated blood glucose and therefore, low probability for hypoglycemia; 2) low primary and secondary failure rates; and 3) preservation of islet cell function.

The present invention provides a novel treatment for diabetes and related disorders by focusing on regulation of the cAMP signaling system by inhibition of phosphodiesterase 10A PDE10A). Phosphodiesterases (PDEs) are a family of cAMP and/or cGMP-hydrolyzing enzymes that cleave 3′,5′-cyclic nucleotide monophosphates to 5′-nucleotide monophosphates. PDEs are known to be involved in the regulation of the cAMP system. Specifically, PDE10A is a phosphodiesterase that hydrolyses cAMP and cGMP with Kₘ values of approximately 0.05-14 μM Fujishige, et al., J. Biol. Chem. 274 (26):18438-18445, 1999; Soderling, et al., PNAS 96:7071-7076, 1999; Loughney, et al., Gene 234:109-117, 1999). At least three splice variants of PDE10A have been described that are identical in their C-terminal catalytic domains, but differ in the size of the N-terminal portion of the molecule (Kotera, et al., Biochem. Biophy. Res. Comm. 261:551-557, 1999; Fujishige, et al., Eur. J. Biochem. 267:5943-5951, 2000).

Thus, by inhibiting PDE10A activity, intracellular levels of cAMP are increased thereby increasing the release of insulin-containing secretory granules and therefore, increasing insulin secretion. The present invention, therefore, provides a novel treatment for diabetes and related disorders, that is, the administration of a PDE10A inhibitor.

### SUMMARY OF THE INVENTION

The present invention relates to methods of treating diabetes, including type 2 diabetes, in a mammal by administering an effective amount of a PDE10A inhibitor. Other methods of the invention relate to treatment of other disorders related to diabetes, such as Syndrome X, impaired glucose tolerance, and impaired fasting glucose, by administering a PDE10A inhibitor. In addition, the present invention relates to methods of treating type 1 diabetes, gestational diabetes, maturity-onset diabetes of the young (MODY), latent autoimmune diabetes adult (LADA), and associated diabetic dyslipidemia and other diabetic complications, as well as hyperglycemia, hyperinsulinemia, dyslipidemia, hypertriglyceridemia, and insulin resistance.

The invention further relates to methods of stimulating insulin release from pancreatic cells in a mammal by administering an effective amount of a PDE10A inhibitor. This method of insulin release may be in response to an elevation of the concentration of glucose in the blood of a mammal. In methods of the invention, the PDE10A inhibitor may also be administered in conjunction with other diabetes therapies, such as alpha-glucosidase inhibitors (e.g., acarbose), insulin sensitizers (e.g., thiazolidinediones), compounds that reduce hepatic glucose output (e.g., metformin), insulin secretagogues (e.g., sulfonylureas), β-3 agonists, and insulin. Furthermore, the PDE10A inhibitor may be administered in conjunction with one or more weight reduction agents, such as Xenical, Meridia, β-3 agonists, or a CB-1 antagonist. Finally, in another embodiment, methods of the invention provide for the administration of a PDE10A inhibitor in combination with an HMG-CoA reductase inhibitor, nicotinic acid, a bile acid sequestrant, a fibric acid derivative, or an antihypertensive drug.

In another aspect of the invention, a PDE10A inhibitor may be administered for the treatment of dementia or a urogenital tract disorder. Urogenital tract disorders include, but are not limited to, incontinence, stress incontinence, benign prostatic hyperplasia, erectile dysfunction, female sexual dysfunction, and hypertrophy of prostate. In further aspect of the invention, a PDE10A inhibitor may be administered for the treatment of a cardiovascular disorder, such as hypertension, ischemic heart disease, myocardial infarction, stable and unstable angina, peripheral occlusive disease, and ischemic stroke.

The present invention, therefore, provides methods for the treatment of diabetes by inhibition of PDE10A through the administration of a PDE10A inhibitor. These and other aspects of the invention will be more apparent from the following description and claims.

### DETAILED DESCRIPTION OF THE INVENTION

Methods of the invention provide for the treatment of diabetes and related disorders, particularly type 2 diabetes, and/or stimulation of insulin release from pancreatic cells, by the administration of a PDE10A inhibitor. Such methods provide for treatment of any condition in which glucose is elevated in the fasting or post-prandial state, by administration of a PDE10A inhibitor. PDE10A has been identified in islets of Langerhans. PDE10A hydrolyses cAMP to AMP and thereby decreases intracellular concentrations of cAMP. By inhibiting PDE10A activity, intracellular levels of cAMP are increased, thereby increasing the release of insulin-containing secretory granules and therefore, increasing insulin secretion. Also as described herein, a PDE10A inhibitor may be administered for the treatment of dementia, cardiovascular disease, or urogenital tract disorders.

Methods of Treatment

As used herein, various terms are defined below.

When introducing elements of the present invention or the preferred embodiment(s) thereof, the articles “a,” “an,” “the,” and “said” are intended to mean that there are one or more of the elements. The terms “comprising,” “including,” and “having” are intended to be inclusive and mean that there may be additional elements other than the listed elements.

The term “subject” as used herein includes mammals (e.g., humans and animals).

The term “treatment” includes any process, action, application, therapy, or the like, wherein a subject, including a human being, is provided medical aid with the object of improving the subject's condition, directly or indirectly, or slowing the progression of a condition or disorder in the subject.

The term “combination therapy” or “co-therapy” means the administration of two or more therapeutic agents to treat a diabetic condition and/or disorder. Such administration encompasses co-administration of two or more therapeutic agents in a substantially simultaneous manner, such as in a single capsule having a fixed ratio of active ingredients or in multiple, separate capsules for each inhibitor agent. In addition, such administration encompasses use of each type of therapeutic agent in a sequential manner.

The phrase “therapeutically effective” means the amount of each agent administered that will achieve the goal of improvement in a diabetic condition or disorder severity, while avoiding or minimizing adverse side effects associated with the given therapeutic treatment.

The term “pharmaceutically acceptable” means that the subject item is appropriate for use in a pharmaceutical product.

Methods of the invention may be used to treat diseases, such as diabetes, including both type 1 and type 2 diabetes. Such methods may also delay the onset of diabetes and diabetic complications. Other diseases and conditions that may be treated or prevented using methods of the invention include: Maturity-Onset Diabetes of the Young (MODY) (Herman, et al., Diabetes 43:40, 1994), Latent Autoimmune Diabetes Adult (LADA) (Zimmet, et al., Diabetes Med. 11:299, 1994), impaired glucose tolerance (IGT) (Expert Committee on Classification of Diabetes Mellitus, Diabetes Care 22 (Supp. 1):S5, 1999), impaired fasting glucose (IFG) (Charles, et al., Diabetes 40:796, 1991), gestational diabetes (Metzger, Diabetes, 40:197, 1991), and metabolic Syndrome X.

Methods of the invention may also be used to treat secondary causes of diabetes (Expert Committee on Classification of Diabetes Mellitus, Diabetes Care 22 (Supp. 1):S5, 1999). Such secondary causes include glucocorticoid excess, growth hormone excess, pheochromocytoma, and drug-induced diabetes. Drugs that may induce diabetes include, but are not limited to, pyriminil, nicotinic acid, glucocorticoids, phenytoin, thyroid hormone, β-adrenergic agents, α-interferon, and drugs used to treat HIV infection.

cAMP-mediated release of insulin is also dependent on the presence of stimulatory glucose concentrations. A method of the invention further relates to stimulating insulin release from islet cells by the administration of a PDE10A inhibitor. Glucose-dependent stimulation of insulin secretion with non-peptide compounds therefore, lowers blood glucose concentrations without causing hypoglycemia.

The methods of the present invention may be used alone or in combination with additional therapies and/or compounds known to those skilled in the art in the treatment of diabetes and related disorders. Alternatively, a PDE10A inhibitor may be used partially or completely, in combination therapy.

For example, a PDE10A inhibitor may be administered in combination with other known therapies for the treatment of diabetes, including PPAR ligands (e.g., agonists, antagonists), insulin secretagogues, for example, sulfonylurea drugs and non-sulfonylurea secretagogues, α-glucosidase inhibitors, insulin sensitizers, hepatic glucose output lowering compounds, insulin and insulin derivatives, and anti-obesity drugs. Such therapies may be administered prior to, concurrently with, or following administration of the compounds of the invention. Insulin and insulin derivatives include both long and short acting forms and formulations of insulin. PPAR ligands may include agonists and/or antagonists of any of the PPAR receptors or combinations thereof. For example, PPAR ligands may include ligands of PPAR-α, PPAR-γ, PPAR-δ or any combination of two or three of the receptors of PPAR. PPAR ligands include, for example, rosiglitazone, troglitazone, and pioglitazone. Sulfonylurea drugs include, for example, glyburide, glimepiride, chlorpropamide, tolbutamide, and glipizide. α-glucosidase inhibitors that may be useful in treating diabetes when administered with a compound of the invention include acarbose, miglitol, and voglibose. Insulin sensitizers that may be useful in treating diabetes include PPAR-γ agonists such as the glitazones (e.g., troglitazone, pioglitazone, englitazone, MCC-555, rosiglitazone, and the like) and other thiazolidinedione and non-thiazolidinedione compounds; biguanides such as metformin and phenformin; protein tyrosine phosphatase-1B (PTP-1B) inhibitors; dipeptidyl peptidase IV (DPP-IV) inhibitors; and 11beta-HSD inhibitors. Hepatic glucose output lowering compounds that may be useful in treating diabetes when administered with a compound of the invention include, for example, glucagon anatgonists and metformin, such as Glucophage and Glucophage XR. Insulin secretagogues that may be useful in treating diabetes when administered with a compound of the invention include sulfonylurea and non-sulfonylurea drugs: GLP-1, GIP, PACAP, secretin, and derivatives thereof; nateglinide, meglitinide, repaglinide, glibenclamide, glimepiride, chlorpropamide, and glipizide. For example, GLP-1 includes derivatives of GLP-1 with longer half-lives than native GLP-1, such as, for example, fatty-acid derivatized GLP-1 and exendin.

A PDE10A inhibitor may also be administered in combination with anti-obesity drugs. Anti-obesity drugs include β-3 agonists; CB-1 antagonists; neuropeptide Y5 inhibitors; Ciliary Neurotrophic Factor and derivatives (e.g., Axokine); appetite suppressants, such as, for example, sibutramine (Meridia); and lipase inhibitors, such as, for example, orlistat (Xenical).

In addition, a PDE10A inhibitor may also be administered in combination with drugs commonly used to treat lipid disorders in diabetic patients. Such drugs include, but are not limited to, HMG-CoA reductase inhibitors, nicotinic acid, fatty acid lowering compounds (e.g., acipimox); lipid lowering drugs (e.g., stanol esters, sterol glycosides such as tiqueside, and azetidinones such as ezetimibe), ACAT inhibitors (such as avasimibe), bile acid sequestrants, bile acid reuptake inhibitors, microsomal triglyceride transport inhibitors, and fibric acid derivatives. HMG-CoA reductase inhibitors include, for example, lovastatin, simvastatin, pravastatin, fluvastatin, atorvastatin, rivastatin, itavastatin, cerivastatin, and ZD-4522. Fibric acid derivatives include, for example, clofibrate, fenofibrate, bezafibrate, ciprofibrate, beclofibrate, etofibrate, and gemfibrozil. Sequestrants include, for example, cholestyramine, colestipol, and dialkylaminoalkyl derivatives of a cross-linked dextran.

Furthermore, a PDE10A inhibitor may also be administered combination with anti-hypertensive drugs, such as, for example, β-blockers and ACE inhibitors. Examples of additional anti-hypertensive agents for use in combination with the compounds of the present invention include calcium channel blockers (L-type and T-type; e.g., diltiazem, verapamil, nifedipine, amlodipine and mybefradil), diuretics (e.g., chlorothiazide, hydrochlorothiazide, flumethiazide, hydroflumethiazide, bendroflumethiazide, methylchlorothiazide, trichloromethiazide, polythiazide, benzthiazide, ethacrynic acid tricrynafen, chlorthalidone, furosemide, musolimine, bumetanide, triamtrenene, amiloride, spironolactone), renin inhibitors, ACE inhibitors (e.g., captopril, zofenopril, fosinopril, enalapril, ceranopril, cilazopril, delapril, pentopril, quinapril, ramipril, lisinopril), AT-1 receptor antagonists (e. g., losartan, irbesartan, valsartan), ET receptor antagonists (e.g., sitaxsentan, atrsentan, neutral endopeptidase (NEP) inhibitors, vasopepsidase inhibitors (dual NEP-ACE inhibitors) (e.g., omapatrilat and gemopatrilat), and nitrates.

Such co-therapies may be administered in any combination of two or more drugs (e.g., a PDE10A inhibitor in combination with an insulin sensitizer and an anti-obesity drug). Such co-therapies may be administered in the form of pharmaceutical compositions, as described below.

Other methods of the invention relate to administration of a PDE10A inhibitor for the treatment of dementia (Shimamoto, et al., Mech. Aging Dev. 5(4):241-250, 1976; Nicholson, et al., Trend Pharmacol. Sci. 12(1):19-27, 1991).

Still further methods of the invention relate to treatment of urogenital tract disorders by the administration of a PDE10A inhibitor. Such urogenital tract disorders include, but are not limited to, incontinence, stress incontinence, benign prostatic hyperplasia, erectile dysfunction, female sexual dysfunction (including female sexual arousal disorder), and hypertrophy of prostate (Ballard, et al., J. Urol. 159(6):2164-2171, 1998).

Other methods of the invention relate to administration of a PDE10A inhibitor to treat cardiovascular disorders, such as hypertension, ischemic heart disease, myocardial infarction, stable and unstable angina, peripheral occlusive disease, and ischemic stroke. Expression of PDE10 can be detected in the heart (Loughney, et al., Gene 234:109-117, 1999; Kotera, et al., Biochem. Biophy. Res. Comm. 261:551-557, 1999), and cGMP and cAMP are important second messengers that are involved in the regulation of vascular smooth muscle tone. The PDE10 family comprises enzymes that are responsible for the degradation of cAMP and cGMP in various tissues (Fujishige, et al., J. Biol. Chem. 274(26):18438-18445, 1999). The activation of soluble and membrane bound guanylate cyclases leads to increased intracellular cGMP levels and induces vasodilation. The stimulation of various G protein-coupled receptors (GPCRs) which are expressed in vascular smooth muscle cells (e.g., adrenomedullin and CGRP receptors) induces the activation of adenylate cyclases, generation of intracellular cAMP, and produces vasodilation. 3′,5′-cyclic nucleotide phosphodiesterases (PDEs) catalyze the hydrolysis of 3′,5′-cyclic nucleotides to their respective nucleoside 5′-monophosphates. Thus, PDE10A likely plays a role in the cardiovascular system.

Pharmaceutical Compositions

Based on well known assays used to determine the efficacy for treatment of conditions identified above in mammals, and by comparison of these results with the results of known medicaments that are used to treat these conditions, the effective dosage of PDE10 inhibitor(s) can readily be determined for treatment of each desired indication. The amount of the active ingredient (e.g., PDE10 inhibitor) to be administered in the treatment of one of these conditions can vary widely according to such considerations as the particular compound and dosage unit employed, the mode of administration, the period of treatment, the age and sex of the patient treated, and the nature and extent of the condition treated.

PDE10A inhibitor(s) for use in methods of the invention may be administered as compound per se. Alternatively, PDE10A inhibitor(s) may be administered with an acceptable carrier in the form of a pharmaceutical composition. The pharmaceutically acceptable carrier must be compatible with the other ingredients of the composition and must not be intolerably deleterious to the recipient. The carrier can be a solid or a liquid, or both, and preferably is formulated with the compound as a unit-dose composition, for example, a tablet, which can contain from about 0.05% to about 95% by weight of the active compound(s) based on a total weight of the dosage form. Other pharmacologically active substances can also be present, including other compounds useful in the treatment of a diabetic condition.

PDE10A inhibitor(s) for use in methods of the present invention may be administered by any suitable route, preferably in the form of a pharmaceutical composition adapted to such a route, and in a therapeutically effective dose for the treatment intended. The PDE10A inhibitor(s) may, for example, be administered orally, sublingually, nasally, pulmonary, mucosally, parenterally, intravascularly, intraperitoneally, subcutaneously, intramuscularly or topically. Unit dose formulations, particularly orally administrable unit dose formulations such as tablets or capsules, generally contain, for example, from about 0.001 to about 500 mg, preferably from about 0.005 mg to about 100 mg, and more preferably from about 0.01 to about 50 mg, of the active ingredient. In the case of pharmaceutically acceptable salts, the weights indicated above for the active ingredient refer to the weight of the pharmaceutically active ion derived from the salt.

Of course, the specific initial and continuing dosage regimen to prevent, treat, give relief from, or ameliorate a diabetic condition or disorder, or to otherwise protect against or treat a diabetic condition for each patient will vary according to the nature and severity of the condition as determined by the attending diagnostician, the activity of the specific PDE10A inhibitor employed, the age of the patient, the diet of the patient, time of administration, route of administration, rate of excretion of the drug, drug combinations, pharmacological considerations such as the activity, efficacy, pharmacokinetics and toxicology profiles of the particular PDE10A inhibitor employed, whether a drug delivery system is utilized, and whether the PDE10A inhibitor is administered with other active ingredients, and the like. The desired mode of treatment and number of doses of a PDE10A inhibitor may be ascertained by those skilled in the art using conventional treatment tests.

The PDE10A inhibitor(s) may be utilized to achieve the desired pharmacological effect by administration to a patient in need thereof in an appropriately formulated pharmaceutical composition. A patient, for the purpose of this invention, is a mammal, including a human, in need of treatment for a particular condition or disease. Therefore, the present invention includes pharmaceutical compositions which are comprised of a pharmaceutically acceptable carrier and a therapeutically effective amount of the PDE10A inhibitor(s). A pharmaceutically acceptable carrier is any carrier which is relatively non-toxic and innocuous to a patient at concentrations consistent with effective activity of the active ingredient so that any side effects ascribable to the carrier do not vitiate the beneficial effects of the active ingredient. The PDE10A inhibitor(s) may be administered with a pharmaceutically-acceptable carrier using any effective conventional dosage unit forms, including, for example, immediate and timed release preparations, orally, parenterally, topically, or the like.

For oral administration, the PDE10A inhibitor(s) may be formulated into solid or liquid preparations such as, for example, capsules, pills, tablets, troches, lozenges, melts, powders, solutions, pastes, syrups, suspensions, or emulsions, and may be prepared according to methods known to the art for the manufacture of pharmaceutical compositions. The solid unit dosage forms may be a capsule which can be of the ordinary hard- or soft-shelled gelatin type containing, for example, surfactants, lubricants, and inert fillers such as lactose, sucrose, calcium phosphate, and corn starch. The pharmaceutical composition is preferably made in the form of a dosage unit containing a particular amount of the active ingredient.

The PDE10A inhibitor(s) may be tableted with conventional tablet bases such as lactose, sucrose, and cornstarch in combination with binders such as acacia, cornstarch, or gelatin; disintegrating agents intended to assist the break-up and dissolution of the tablet following administration such as potato starch, alginic acid, corn starch, and guar gum; lubricants intended to improve the flow of tablet granulation and to prevent the adhesion of tablet material to the surfaces of the tablet dies and punches, for example, talc, stearic acid, or magnesium, calcium or zinc stearate; dyes; coloring agents; and flavoring agents intended to enhance the aesthetic qualities of the tablets and make them more acceptable to the patient. Suitable excipients for use in oral liquid dosage forms include diluents such as water and alcohols, for example, ethanol, benzyl alcohol, and polyethylene alcohols, either with or without the addition of a pharmaceutically acceptable surfactant, suspending agent, or emulsifying agent. Various other materials may be present as coatings or to otherwise modify the physical form of the dosage unit. For instance tablets, pills or capsules may be coated with shellac, sugar or both.

Dispersible powders and granules are suitable for the preparation of an aqueous suspension. They provide the active ingredient in admixture with a dispersing or wetting agent, a suspending agent, and one or more preservatives. Suitable dispersing or wetting agents and suspending agents are exemplified by those already mentioned above. Additional excipients, for example, those sweetening, flavoring and coloring agents described above, may also be present.

The pharmaceutical compositions of this invention may also be in the form of oil-in-water emulsions. The oily phase may be a vegetable oil such as liquid paraffin or a mixture of vegetable oils. Suitable emulsifying agents may be (1) naturally occurring gums such as gum acacia and gum tragacanth, (2) naturally occurring phosphatides such as soy bean and lecithin, (3) esters or partial esters derived from fatty acids and hexitol anhydrides, for example, sorbitan monooleate, and (4) condensation products of said partial esters with ethylene oxide, for example, polyoxyethylene sorbitan monooleate. The emulsions may also contain sweetening and flavoring agents.

Oily suspensions may be formulated by suspending the active ingredient in a vegetable oil such as, for example, arachis oil, olive oil, sesame oil, or coconut oil; or in a mineral oil such as liquid paraffin. The oily suspensions may contain a thickening agent such as, for example, beeswax, hard paraffin, or cetyl alcohol. The suspensions may also contain one or more preservatives, for example, ethyl or n-propyl p-hydroxybenzoate; one or more coloring agents; one or more flavoring agents; and one or more sweetening agents such as sucrose or saccharin.

Syrups and elixirs may be formulated with sweetening agents such as, for example, glycerol, propylene glycol, sorbitol, or sucrose. Such formulations may also contain a demulcent, and preservative, flavoring and coloring agents.

Oral delivery of the PDE10A inhibitor(s) can include formulations well known in the art to provide immediate delivery or prolonged or sustained delivery of a drug to the gastrointestinal tract by any number of mechanisms. Immediate delivery formulations include, but are not limited to, oral solutions, oral suspensions, fast-dissolving tablets or capsules, sublingual tablets, disintegrating tablets and the like. Prolonged or sustained delivery formulations include, but are not limited to, pH sensitive release of the active ingredient from the dosage form based on the changing pH of the small intestine, slow erosion of a tablet or capsule, retention in the stomach based on the physical properties of the formulation, bioadhesion of the dosage form to the mucosal lining of the intestinal tract, or enzymatic release of the active drug from the dosage form. The intended effect is to extend the time period over which an active drug molecule is delivered to the site of action by manipulation of the dosage form. Thus, enteric-coated and enteric-coated controlled release formulations may be used in methods of the present invention. Suitable enteric coatings include cellulose acetate phthalate, polyvinylacetate phthalate, hydroxypropylmethyl-cellulose phthalate and anionic polymers of methacrylic acid and methacrylic acid methyl ester.

Pharmaceutical compositions can be prepared by any suitable method of pharmacy, which includes the step of bringing into association, the PDE10A inhibitor(s) and the carrier (which can constitute one or more accessory ingredients). In general, the compositions are prepared by uniformly and intimately admixing the PDE10A inhibitor(s) with a liquid or finely divided solid carrier, or both, and then, if necessary, shaping the product. For example, a tablet can be prepared by compressing or molding a powder or granules of the inhibitors, optionally with one or more accessory ingredients. Compressed tablets can be prepared by compressing, in a suitable machine, the compound in a free-flowing form, such as a powder or granules optionally mixed with a binder, lubricant, inert diluent and/or surface active/dispersing agent(s). Molded tablets can be made, for example, by molding the powdered compound in a suitable machine.

Liquid dosage forms for oral administration can include pharmaceutically acceptable emulsions, solutions, suspensions, syrups, and elixirs containing inert diluents commonly used in the art, such as water. Such compositions may also comprise adjuvants, such as wetting agents, emulsifying and suspending agents, and sweetening, flavoring, and perfuming agents.

Pharmaceutical compositions suitable for buccal (sub-lingual) administration include lozenges comprising a PDE10A inhibitor in a flavored base, usually sucrose, and acacia or tragacanth, and pastilles comprising the inhibitors in an inert base such as gelatin and glycerin or sucrose and acacia.

The PDE10A inhibitor(s) may also be administered parenterally, that is, subcutaneously, intravenously, intramuscularly, or interperitoneally, as injectable dosages of the compound in a physiologically acceptable diluent with a pharmaceutical carrier which may be a sterile liquid or mixture of liquids such as water, saline, aqueous dextrose and related sugar solutions; an alcohol such as ethanol, isopropanol, or hexadecyl alcohol; glycols such as propylene glycol or polyethylene glycol; glycerol ketals such as 2,2-dimethyl-1,1-dioxolane-4-methanol, ethers such as poly(ethyleneglycol) 400; an oil; a fatty acid; a fatty acid ester or glyceride; or an acetylated fatty acid glyceride with or without the addition of a pharmaceutically acceptable surfactant such as a soap or a detergent, suspending agent such as pectin, carbomers, methycellulose, hydroxypropylmethylcellulose, or carboxymethylcellulose, or emulsifying agent and other pharmaceutical adjuvants.

Illustrative of oils which can be used in the parenteral formulations of this invention are those of petroleum, animal, vegetable, or synthetic origin, for example, peanut oil, soybean oil, sesame oil, cottonseed oil, corn oil, olive oil, petrolatum, and mineral oil. Suitable fatty acids include oleic acid, stearic acid, and isostearic acid. Suitable fatty acid esters are, for example, ethyl oleate and isopropyl myristate. Suitable soaps include fatty alkali metal, ammonium, and triethanolamine salts and suitable detergents include cationic detergents, for example, dimethyl dialkyl ammonium halides, alkyl pyridinium halides, and alkylamine acetates; anionic detergents, for example, alkyl, aryl, and olefin sulfonates, alkyl, olefin, ether, and monoglyceride sulfates, and sulfosuccinates; nonionic detergents, for example, fatty amine oxides, fatty acid alkanolamides, and polyoxyethylenepolypropylene copolymers; and amphoteric detergents, for example, alkyl-beta-aminopropionates, and 2-alkylimidazoline quarternary ammonium salts, as well as mixtures.

The parenteral compositions of this invention may typically contain from about 0.5% to about 25% by weight of the active ingredient in solution. Preservatives and buffers may also be used advantageously. In order to minimize or eliminate irritation at the site of injection, such compositions may contain a non-ionic surfactant having a hydrophile-lipophile balance (HLB) of from about 12 to about 17. The quantity of surfactant in such formulation ranges from about 5% to about 15% by weight. The surfactant can be a single component having the above HLB or can be a mixture of two or more components having the desired HLB.

Illustrative of surfactants used in parenteral formulations are the class of polyethylene sorbitan fatty acid esters, for example, sorbitan monooleate and the high molecular weight adducts of ethylene oxide with a hydrophobic base, formed by the condensation of propylene oxide with propylene glycol.

The pharmaceutical compositions may be in the form of sterile injectable aqueous suspensions. Such suspensions may be formulated according to known methods using suitable dispersing or wetting agents and suspending agents such as, for example, sodium carboxymethylcellulose, methylcellulose, hydroxypropylmethyl-cellulose, sodium alginate, polyvinylpyrrolidone, gum tragacanth and gum acacia; dispersing or wetting agents which may be a naturally occurring phosphatide such as lecithin, a condensation product of an alkylene oxide with a fatty acid, for example, polyoxyethylene stearate, a condensation product of ethylene oxide with a long chain aliphatic alcohol, for example, heptadecaethyleneoxycetanol, a condensation product of ethylene oxide with a partial ester derived form a fatty acid and a hexitol such as polyoxyethylene sorbitol monooleate, or a condensation product of an ethylene oxide with a partial ester derived from a fatty acid and a hexitol anhydride, for example polyoxyethylene sorbitan monooleate.

The sterile injectable preparation may also be a sterile injectable solution or suspension in a non-toxic parenterally acceptable diluent or solvent. Diluents and solvents that may be employed are, for example, water, Ringer's solution, and isotonic sodium chloride solution. In addition, sterile fixed oils are conventionally employed as solvents or suspending media. For this purpose, any bland, fixed oil may be employed including synthetic mono or diglycerides. In addition, fatty acids such as oleic acid may be used in the preparation of injectables.

A composition of the invention may also be administered in the form of suppositories for rectal administration of the drug. These compositions may be prepared by mixing the drug (e.g., PDE10A inhibitor) with a suitable non-irritation excipient which is solid at ordinary temperatures but liquid at the rectal temperature and will therefore melt in the rectum to release the drug. Such material are, for example, cocoa butter and polyethylene glycol.

Another formulation employed in the methods of the present invention employs transdermal delivery devices (“patches”). Such transdermal patches may be used to provide continuous or discontinuous infusion of the compounds of the present invention in controlled amounts. The construction and use of transdermal patches for the delivery of pharmaceutical agents is well known in the art (see, e.g., U.S. Pat. No. 5,023,252, incorporated herein by reference). Such patches may be constructed for continuous, pulsatile, or on demand delivery of pharmaceutical agents.

It may be desirable or necessary to introduce the pharmaceutical composition to the patient via a mechanical delivery device. The construction and use of mechanical delivery devices for the delivery of pharmaceutical agents is well known in the art. For example, direct techniques for administering a drug directly to the brain usually involve placement of a drug delivery catheter into the patient's ventricular system to bypass the blood-brain barrier. One such implantable delivery system, used for the transport of agents to specific anatomical regions of the body, is described in U.S. Pat. No. 5,011,472, incorporated herein by reference.

The compositions of the invention may also contain other conventional pharmaceutically acceptable compounding ingredients, generally referred to as carriers or diluents, as necessary or desired. Any of the compositions of this invention may be preserved by the addition of an antioxidant such as ascorbic acid or by other suitable preservatives. Conventional procedures for preparing such compositions in appropriate dosage forms can be utilized.

Commonly used pharmaceutical ingredients which may be used as appropriate to formulate the composition for its intended route of administration include: acidifying agents, for example, but are not limited to, acetic acid, citric acid, fumaric acid, hydrochloric acid, nitric acid; and alkalinizing agents such as, but are not limited to, ammonia solution, ammonium carbonate, diethanolamine, monoethanolamine, potassium hydroxide, sodium borate, sodium carbonate, sodium hydroxide, triethanolamine, trolamine.

Other pharmaceutical ingredients include, for example, but are not limited to, adsorbents (e.g., powdered cellulose and activated charcoal); aerosol propellants (e.g., carbon dioxide, CCl₂F₂, F2ClC—CClF₂ and CClF₃); air displacement agents (e.g., nitrogen and argon); antifungal preservatives (e.g., benzoic acid, butylparaben, ethylparaben, methylparaben, propylparaben, sodium benzoate); antimicrobial preservatives (e.g., benzalkonium chloride, benzethonium chloride, benzyl alcohol, cetylpyridinium chloride, chlorobutanol, phenol, phenylethyl alcohol, phenylmercuric nitrate and thimerosal); antioxidants (e.g., ascorbic acid, ascorbyl palmitate, butylated hydroxyanisole, butylated hydroxytoluene, hypophosphorus acid, monothioglycerol, propyl gallate, sodium ascorbate, sodium bisulfite, sodium formaldehyde sulfoxylate, sodium metabisulfite); binding materials (e.g., block polymers, natural and synthetic rubber, polyacrylates, polyurethanes, silicones and styrene-butadiene copolymers); buffering agents (e.g., potassium metaphosphate, potassium phosphate monobasic, sodium acetate, sodium citrate anhydrous and sodium citrate dihydrate); carrying agents (e.g., acacia syrup, aromatic syrup, aromatic elixir, cherry syrup, cocoa syrup, orange syrup, syrup, corn oil, mineral oil, peanut oil, sesame oil, bacteriostatic sodium chloride injection and bacteriostatic water for injection); chelating agents (e.g., edetate disodium and edetic acid); colorants (e.g., FD&C Red No. 3, FD&C Red No. 20, FD&C Yellow No. 6, FD&C Blue No. 2, D&C Green No. 5, D&C Orange No. 5, D&C Red No. 8, caramel and ferric oxide red); clarifying agents (e.g., bentonite); emulsifying agents (but are not limited to, acacia, cetomacrogol, cetyl alcohol, glyceryl monostearate, lecithin, sorbitan monooleate, polyethylene 50 stearate); encapsulating agents (e.g., gelatin and cellulose acetate phthalate); flavorants (e.g., anise oil, cinnamon oil, cocoa, menthol, orange oil, peppermint oil and vanillin); humectants (e.g., glycerin, propylene glycol and sorbitol); levigating agents (e.g., mineral oil and glycerin); oils (e.g., arachis oil, mineral oil, olive oil, peanut oil, sesame oil and vegetable oil); ointment bases (e.g., lanolin, hydrophilic ointment, polyethylene glycol ointment, petrolatum, hydrophilic petrolatum, white ointment, yellow ointment, and rose water ointment); penetration enhancers (transdermal delivery) (e.g., monohydroxy or polyhydroxy alcohols, saturated or unsaturated fatty alcohols, saturated or unsaturated fatty esters, saturated or unsaturated dicarboxylic acids, essential oils, phosphatidyl derivatives, cephalin, terpenes, amides, ethers, ketones and ureas); plasticizers (e.g., diethyl phthalate and glycerin); solvents (e.g., alcohol, corn oil, cottonseed oil glycerin, isopropyl alcohol, mineral oil, oleic acid, peanut oil, purified water, water for injection, sterile water for injection and sterile water for irrigation); stiffening agents (e.g., cetyl alcohol, cetyl esters wax, microcrystalline wax, paraffin, stearyl alcohol, white wax and yellow wax); suppository bases (e.g., cocoa butter and polyethylene glycols (mixtures)); surfactants (e.g., benzalkonium chloride, nonoxynol 10, oxtoxynol 9, polysorbate 80, sodium lauryl sulfate and sorbitan monopalmitate); suspending agents (e.g., agar, bentonite, carbomers, carboxymethylcellulose sodium, hydroxyethyl cellulose, hydroxypropyl cellulose, hydroxypropyl methylcellulose, kaolin, methylcellulose, tragacanth and veegum); sweetening e.g., aspartame, dextrose, glycerin, mannitol, propylene glycol, saccharin sodium, sorbitol and sucrose); tablet anti-adherents (e.g., magnesium stearate and talc); tablet binders (e.g., acacia, alginic acid, carboxymethylcellulose sodium, compressible sugar, ethylcellulose, gelatin, liquid glucose, methylcellulose, povidone and pregelatinized starch); tablet and capsule diluents (e.g., dibasic calcium phosphate, kaolin, lactose, mannitol, microcrystalline cellulose, powdered cellulose, precipitated calcium carbonate, sodium carbonate, sodium phosphate, sorbitol and starch); tablet coating agents (e.g., liquid glucose, hydroxyethyl cellulose, hydroxypropyl cellulose, hydroxypropyl methylcellulose, methylcellulose, ethylcellulose, cellulose acetate phthalate and shellac); tablet direct compression excipients (e.g., dibasic calcium phosphate); tablet disintegrants (e.g., alginic acid, carboxymethylcellulose calcium, microcrystalline cellulose, polacrillin potassium, sodium alginate, sodium starch glycollate and starch); tablet glidants (e.g., colloidal silica, corn starch and talc); tablet lubricants (e.g., calcium stearate, magnesium stearate, mineral oil, stearic acid and zinc stearate); tablet/capsule opaquants (e.g., titanium dioxide); tablet polishing agents (e.g., carnuba wax and white wax); thickening agents (e.g., beeswax, cetyl alcohol and paraffin); tonicity agents (e.g., dextrose and sodium chloride); viscosity increasing agents (e.g., alginic acid, bentonite, carbomers, carboxymethylcellulose sodium, methylcellulose, povidone, sodium alginate and tragacanth); and wetting agents (e.g., heptadecaethylene oxycetanol, lecithins, polyethylene sorbitol monooleate, polyoxyethylene sorbitol monooleate, and polyoxyethylene stearate).

Dosage levels of the PDE10A inhibitors for use in methods of this invention typically are from about 0.001 mg to about 10,000 mg daily, preferably from about 0.005 mg to about 1,000 mg daily. On the basis of mg/kg daily dose, either given in a single or divided doses, dosages typically range from about 0.001/75 mg/kg to about 10,000/75 mg/kg, preferably from about 0.005/75 mg/kg to about 1,000/75 mg/kg.

The total daily dose of each inhibitor can be administered to the patient in a single dose, or in multiple subdoses. Typically, subdoses can be administered two to six times per day, preferably two to four times per day, and even more preferably two to three times per day. Doses can be in immediate release form or sustained release form sufficiently effective to obtain the desired control over the diabetic condition.

The PDE10A inhibitor(s) may be administered as the sole pharmaceutical agent or in combination with one or more other pharmaceutical agents where the combination causes no unacceptable adverse effects. For example, the PDE10A inhibitor(s) can be combined with known anti-obesity, or with known antidiabetic or other indication agents, and the like, as well as with admixtures and combinations thereof

The PDE10A inhibitor(s) may also be utilized in compositions, in research and diagnostics, or as analytical reference standards, and the like. Therefore, the present invention includes compositions which are comprised of an inert carrier and an effective amount of the PDE10A inhibitor(s). An inert carrier is any material which does not interact with a compound to be carried and which lends support, means of conveyance, bulk, traceable material, and the like to the compound to be carried. An effective amount of compound is that amount which produces a result or exerts an influence on the particular procedure being performed.

A PDE10A inhibitor for use in methods of the invention may also be administered as the pharmaceutically acceptable salt, protected acid, conjugate acid, tautomer, prodrug or stereoisomer of a compound found to inhibit the activity of PDE10A. Tautomers include, for example, hydroxy tautomers. Protected acids include, but are not limited to, protected acids such as esters, hydroxyamino derivatives, amides and sulfonamides. Formation of prodrugs is well known in the art in order to enhance the properties of the parent compound; such properties include solubility, absorption, biostability and release time (see “Pharmaceutical Dosage Form and Drug Delivery Systems” (Sixth Edition), edited by Ansel et al., publ. by Williams & Wilkins, pgs. 27-29, (1995) which is hereby incorporated by reference). Commonly used prodrugs are designed to take advantage of the major drug biotransformation reactions and are also to be considered within the scope of the invention. Major drug biotransformation reactions include N-dealkylation, O-dealkylation, aliphatic hydroxylation, aromatic hydroxylation, N-oxidation, S-oxidation, deamination, hydrolysis reactions, glucuronidation, sulfation and acetylation (see Goodman and Gilman's The Pharmacological Basis of Therapeutics (Ninth Edition), editor Molinoff et al., publ. by McGraw-Hill, pages 11-13, (1996), which is hereby incorporated by reference).

Besides being useful for human treatment, administration of a PDE10A inhibitor may also be useful for veterinary treatments of companion animals (e.g., horses, dogs, cats, etc.), exotic animals and farm animals. Even though the invention is described in terms of human biology, it is understood by those of ordinary skill in the art that the present invention is applicable to other mammals as well.

Formulations suitable for subcutaneous, intravenous, intramuscular, and the like; suitable pharmaceutical carriers; and techniques for formulation and administration may be prepared by any of the methods well known in the art (see, e.g., Remington's Pharmaceutical Sciences, Mack Publishing Co., Easton, Pa., 20^{th} edition, 2000).

It should be apparent to one of ordinary skill in the art that changes and modifications can be made to this invention without departing from the spirit or scope of the invention as it is set forth herein.

### EXAMPLES

In order that this invention may be better understood, the following examples are set forth. These examples are for the purpose of illustration only, and are not to be construed as limiting the scope of the invention in any manner. All publications mentioned herein are incorporated by reference in their entirety.

Demonstration of the activity of the compounds of the present invention may be accomplished through in vitro, ex vivo, and in vivo assays that are well known in the art. For example, to demonstrate the efficacy of a pharmaceutical agent for the treatment of diabetes and related disorders, the following assays may be used.

Expression Profiling

Quantitative PCR Expression Analysis

The expression of PDE10A in INS-1E (44) cell-line and dispersed islets was verified by PCR.

RNA Extraction and cDNA Preparation

Total RNA for TaqMan quantitative analysis was extracted from cells according to the vendor protocol that utilizes the RNeasy protocol for isolation of total RNA from animal cells (Qiagen, Valencia, Calif.).

RNA (100 μg) was treated with DNase I using RNase free-DNase (Qiagen, Valencia, Calif.). After elution and quantitation on an Agilent 2100 Bioanalyzer (Agilent Technologies, Palo Alto, Calif.), each sample was reverse transcribed using the Superscript II First Strand Synthesis System for RT-PCR according to the vendor protocol (Invitrogen, Carlsbad, Calif.). The final concentration of RNA in the reaction mix was 50 ng/μL.

TaqMan Quantitative Analysis

Specific primers and probes were designed according to Applied Biosystems (Foster City, Calif.) Assays-By-Design service and are listed below:

TaqMan Primer Sequences:

| | |
|---|---|
| PDE10A-forward | 5′-(CAGATCCTCCCACCCACAGA)-3′ |
| primer: | |
| PDE10A-reverse | 5′-(TCGAATTACCTTCTCCCACTGATT)-3′ |
| primer: | |
| PDE10A-probe: | 5′-(FAM)-(TGCAGGGATAACCTC) |
| | (MGB)-3′ |

where FAM=6-carboxy-fluorescein and MGB=minor groove binder. The expected length of the PCR product was 75 bp.

Quantitation experiments were performed on reverse transcribed RNA (25 ng) from each sample. Ribosomal RNA (18S) was measured as a control using the Pre-Developed TaqMan Assay Reagents (PDAR) (Applied Biosystems, Foster City, Calif.).

The assay reaction mix was as follows:

| | final |
|---|---|
| TaqMan Universal PCR Master Mix (2x) | 1x |
| PDAR control - 18S RNA (20x) | 1x |
| Forward primer | 300 nM |
| Reverse primer | 300 nM |
| Probe | 200 nM |
| cDNA | 25 ng |
| Water | to 25 μL |

PCR Conditions:

| | |
|---|---|
| One cycle: | 2 minutes at 50° C. |
| | 10 minutes at 95° C. |
| 40 cycles: | 15 seconds at 95° C. |
| | 1 minute at 60° C. |

The experiment was performed on an ABI Prism 7700 Sequence Detector (Applied Biosystems, Foster City, Calif.). At the end of the run, fluorescence data acquired during PCR were processed as described in the ABI Prism 7700 user's manual.

Table 1 shows the PDE10A and 18S housekeeping CT values using the primer combinations described above. The CT value is the threshold cycle which product fluorescence rises above background. CT values>35 indicate the gene is not expressed; CT values of 30 to 35 indicate low expression of the gene; CT values of 25 to 30 indicate expression; and CT values<25 indicate high expression. Data presented in Table 1 is representative of three determinations. As shown in Table 1, PDE10A is expressed in the INS-1E (44) cell-line and dispersed islet cells. Expression of PDE10A in islet cells indicates that PDE10A may have a role in regulating insulin release/blood glucose concentrations. NTC=no template control.

TABLE 1

| | PDE10A | 18S |
|---|---|---|
| Sample Name | (CT) | (CT) |
| INS-1 (E44) | 24.56 | 14.27 |
| Dispersed islets | 29.51 | 17.1 |
| NTC | 40 | 39.44 |

PDE10A Inhibition Assay

Test compounds and PDE10A enzyme in assay buffer are added to 96-well whitewall/clear bottom isoplates (Wallac). The reaction is initiated by the addition of ³H-cAMP (Amersham) or ³H-cGMP (Amersham). Following a 45-minute incubation at room temperature, the reaction is stopped by the addition of SPA yttrium silicate beads (Amersham). The samples are incubated for an additional 30 minutes, and the plates are read in the Microbeta (Wallac) for 30 seconds in the SPA mode. Data may be expressed as a percentage of control. To measure PDE2, PDE3A, PDE4B, and PDE10A activity, 3H-cAMP may be used as a substrate; and to measure PDE5 activity, 3H-cGMP may be used as a substrate.

Compounds were identified that inhibited the activity of PDE10A with an IC₅₀ value of 0.1 μM or less (see, e.g., WO 02/048144 and WO 03/014116, incorporated by reference in its entirety). For example, the compounds include the following:

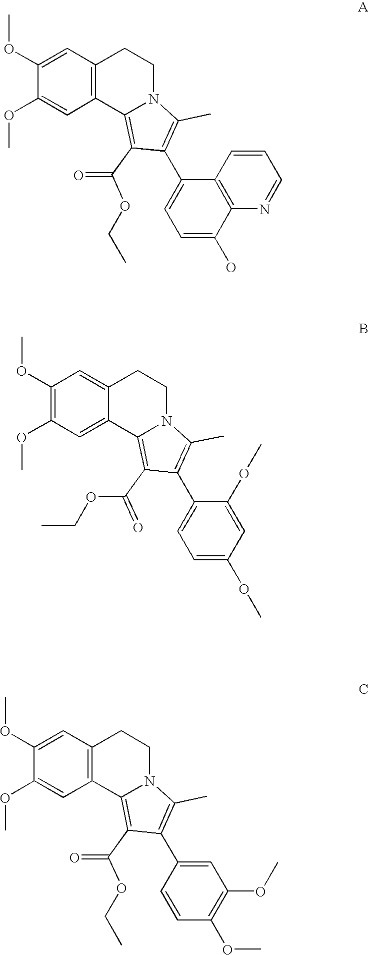

These same compounds were run in inhibition assays for PDE2, PDE3A, PDE4B, and PDE5. In these assays, the compounds were found to have IC₅₀ values that were at least 100-fold greater than the IC₅₀ value for PDE10A (Table 2). The compounds are therefore selective for PDE10A.

TABLE 2

| | PDE10A | PDE2 | PDE3 | PDE4B | PDE5 |
|---|---|---|---|---|---|
| Compound | nM | μM | μM | μM | μM |
| A | 30 | 10 | >10 | 10 | 10 |
| B | 32.7 | >10 | >10 | 10 | >10 |
| C | 26.3 | >10 | >10 | >10 | >10 |

Dispersed Islet Assay

Dispersed pancreatic islet isolation. Lean rats (male Sprague-Dawley, 200-250 g) are anesthetized with nembutal (60 mg/kg, i.p.) and the abdomen opened to expose the liver and pancreas. The pancreas is distended by injection of Hank's solution into the bile duct, and then the pancreas is excised and minced with scissors while in Hank's solution. After rinsing the tissue with buffer, the pancreas is digested for 10 minutes with collagenase, rinsed, and the islets separated from debris on a Ficoll gradient. The isolated islet fraction is then rinsed with buffer, incubated with EDTA for 8 minutes, followed by incubation with trypsin and DNAase I for an additional 10 minutes. The dispersed islets are transferred to culture media containing 8 mM glucose, seeded in “V-bottom” 96-well plates (2,500 cells per well), and cultured overnight.

For assaying insulinotropic compounds, the dispersed islets are pre-incubated in 3 mM glucose for 30 minutes. The islets are then transferred to media containing 8 mM glucose, test compounds, and 0.3 μM forskolin; and incubated for an additional 30 minutes. The media is then assayed for insulin content using, for example, a SPA assay. Insulin secretion is reported as fold over control (FOC), where the control is the amount of insulin secretion in the presence of 8 mM glucose and 0.3 μM forskolin. Results for Compound A, B, and C may be found in Table 3.

TABLE 3

| | Fold-over-Control |
|---|---|
| Compound | (FOC) |
| A | 1.4 |
| B | 1.44 |
| C | 1.48 |

In Vivo Assay

Lean rats (male Wistar, 250-300 g) are fasted overnight and divided into two groups: vehicle and compound treatment (8 rats per group). Vehicle or compound is administrated via oral gavage (1.5 mL/rat). Two hours later, a glucose solution (30%, 2 g/kg body weight) is injected intraperitoneally. Tail blood samples are collected at 0, 15, 30, and 60-minute time points after the glucose injection, and blood glucose is measured using Glucometer (Bayer Diagnostics, Mishawaka, Ind.).

All publications and patents mentioned in the above specification are incorporated herein by reference. Various modifications and variations of the described methods of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the above-described modes for carrying out the invention which are obvious to those skilled in the field of diabetes or related fields are intended to be within the scope of the following claims. Those skilled in the art will recognize, or be able to ascertain using no more than routine experimentation, many equivalents to the specific embodiments of the invention described herein. Such equivalents are intended to be encompassed by the following claims.

What is claimed is:

1. A method of treating or preventing a disease or condition selected from the group consisting of diabetes, maturity-onset diabetes of the young (MODY), latent autoimmune diabetes adult (LADA), impaired glucose tolerance (IGT), impaired fasting glucose (IFG), gestational diabetes, and metabolic syndrome X, comprising administering to a mammal an effective amount of a PDE10A inhibitor.

2. The method of claim 1, wherein diabetes is type 2 diabetes.

3. The method of claim 1, further comprising administering insulin, insulin derivatives, PPAR ligands, sulfonylurea drugs, α-glucosidase inhibitors, biguanides, PTP-1B inhibitors, DPP-IV inhibitors, 11-beta-HSD inhibitors, GLP-1 and GLP-1 derivatives, GIP and GIP derivatives, PACAP and PACAP derivatives, or secretin and secretin derivatives in combination with said PDE10A inhibitor.

4. The method of claim 3, wherein said PPAR ligand is selected from rosiglitazone, troglitazone, and pioglitazone.

5. The method of claim 3, wherein said sulfonylurea is selected from glibenclamide, glimepiride, chlorpropamide, glipizide, glyburide, and tolbutamide.

6. The method of claim 3, wherein said α-glucosidase inhibitor is selected from acarbose, miglitol, and voglibose.

7. The method of claim 1, further comprising administering HMG-CoA inhibitors, nicotinic acid, fatty acid lowering compounds, lipid lowering drugs, ACAT inhibitors, bile sequestrants, bile acid reuptake inhibitors, microsomal triglyceride transport inhibitors, or fibric acid derivatives in combination with said PDE10A inhibitor.

8. The method of claim 1, further comprising administering an anti-obesity agent selected from the group consisting of β-3 agonists, CB-1 antagonists, neuropeptide Y5 inhibitors, appetite suppressants, and lipase inhibitors in combination with said PDE10A inhibitor.

9. The method of claim 1, further comprising administering an anti-hypertensive agent selected from the group consisting of β-blockers, calcium channel blockers, diuretics, renin inhibitors, ACE inhibitors, AT-1 receptor antagonists, ET receptor antagonists, and nitrates in combination with said PDE10A inhibitor.

10. A method of treating or preventing secondary causes of diabetes selected from glucocorticoid excess, growth hormone excess, pheochromocytoma, and drug-induced diabetes, comprising administering to a mammal an effective amount of a PDE10A inhibitor.

11. A method of increasing the sensitivity of pancreatic β-cells to an insulin secretagogue, comprising administering to a mammal an effective amount of a PDE10A inhibitor.

12. The method of claim 11, wherein said insulin secretagogue is selected from GLP-1, GIP, PAC/VPAC receptor agonists, secretin, nateglinide, meglitinide, repaglinide, glibenclamide, glimepiride, chlorpropamide, and glipizide.

13. A method of treating or preventing dementia, comprising administering to a mammal an effective amount of a PDE10A inhibitor.

14. A method of treating or preventing a cardiovascular disorder selected from hypertension, ischemic heart disease, myocardial infarction, stable and unstable angina, peripheral occlusive disease and ischemic stroke, comprising administering to a mammal an effective amount of a PDE10A inhibitor.

15. A method of treating or preventing a urogenital tract disorder selected from incontinence, stress incontinence, benign prostatic hyperplasia, erectile dysfunction, female sexual dysfunction, and prostatic hypertrophy, comprising administering to a mammal an effective amount of a PDE10A inhibitor.

16. The method of claim 16, wherein said female sexual dysfunction is female sexual arousal disorder.

17. A method for identifying compounds which are useful for the treatment of diabetes, maturity-onset diabetes of the young (MODY), latent autoimmune diabetes adult (LADA), impaired glucose tolerance (IGT), impaired fasting glucose (IFG), gestational diabetes, metabolic syndrome X, dementia, cardiovascular disorders, and urogenital tract disorders, comprising the step of determining whether the compound inhibits PDE10A.

18. A method for the treatment of diabetes, maturity-onset diabetes of the young (MODY), latent autoimmune diabetes adult (LADA), impaired glucose tolerance (IGT), impaired fasting glucose (IFG), gestational diabetes, metabolic syndrome X, dementia, cardiovascular disorders, and urogenital tract disorders, comprising administering to a mammal an effective amount of a compound identified by the method of claim 17.

20. A pharmaceutical composition comprising a therapeutically effective amount of a compound which inhibits PDE10A in combination with a pharmaceutically acceptable carrier.

21. A pharmaceutical composition comprising a therapeutically effective amount of a compound which inhibits PDE10A in combination with a pharmaceutically acceptable carrier and one or more pharmaceutical agents.

22. The pharmaceutical composition of claim 21, wherein said pharmaceutical agent is selected from the group consisting of insulin, insulin derivatives, PPAR ligands, sulfonylurea drugs, α-glucosidase inhibitors, biguanides, PTP-1B inhibitors, DPP-IV inhibitors, 11-beta-HSD inhibitors, GLP-1 and GLP-1 derivatives, GIP and GIP derivatives, PACAP and PACAP derivatives, and secretin and secretin derivatives.

23. The pharmaceutical composition of claim 21, wherein said pharmaceutical agent is selected from the group consisting of β-3 agonists, CB-1 antagonists, neuropeptide Y5 inhibitors, appetite suppressants, and lipase inhibitors.

24. The pharmaceutical composition of claim 21, wherein said pharmaceutical agent is selected from the group consisting of HMG-CoA inhibitors, nicotinic acid, fatty acid lowering compounds, lipid lowering drugs, ACAT inhibitors, bile sequestrants, bile acid reuptake inhibitors, microsomal triglyceride transport inhibitors, and fibric acid derivatives.

25. The pharmaceutical composition of claim 21, wherein said pharmaceutical agent is an anti-hypertensive agent selected from the group consisting of β-blockers, calcium channel blockers, diuretics, renin inhibitors, ACE inhibitors, AT-1 receptor antagonists, ET receptor antagonists, and nitrates.


SEQUENCE LISTING (3 sequences)

SEQ ID NO: 1 - DNA, 20 bp, Artificial
  Primer
cagatcctcccacccacaga

SEQ ID NO: 2 - DNA, 24 bp, Artificial
  Primer
tcgaattaccttctcccactgatt

SEQ ID NO: 3 - DNA, 15 bp, Artificial
  Probe
tgcagggataacctc